(19) United States
(12) Patent Application Publication
(10) Pub. No.: US 2016/0199282 A1
(43) Pub. Date: Jul. 14, 2016

(54) PERFORMANCE-ENHANCED HAIR TREATMENT COMPOSITION

(71) Applicant: Henkel AG & Co. KGaA, Duesseldorf (DE)

(72) Inventors: Erik Schulze zur Wiesche, Hamburg (DE); Monkia Nebel, Norderstedt (DE)

(73) Assignee: Henkel AG & Co. KGaA, Duesseldorf (DE)

(21) Appl. No.: 15/076,795

(22) Filed: Mar. 22, 2016

Related U.S. Application Data
(63) Continuation of application No. PCT/DE2014/200418, filed on Aug. 22, 2014.

(30) Foreign Application Priority Data
Oct. 1, 2013 (DE) 10 2013 219 919.8

(51) Int. Cl. A61K 8/65; A61Q 5/12; A61K 8/64

(52) U.S. Cl. CPC A61K 8/65; A61K 8/64; A61Q 5/12; A61K 2800/5426; A61K 2800/594

(57) ABSTRACT

Hair treatment compositions include—based in each case on the weight thereof—0.0001% to 10% by weight at least one oligopeptide having at least one Glu-Glu-Glu amino acid sequence, wherein the amino group may be free or protonated and the carboxyl groups may be free or deprotonated, and 0.0001% to 10% by weight at least one proteolipid of the formula (I) in which R1 is a straight-chain hydrocarbyl radical having 8 to 22 carbon atoms, R2 is (—CH₂)_{y}—CH₃, with y=0 to 22, Y is —H or —OH, X− is a physiologically acceptable anion, Hy is an oligopeptide residue from keratin hydrolysate, and x has values from 0 to 22. In these compositions, proteolipids and oligopeptides have mutually enhancing positive effects on the hair and the hair follicle.

### FIELD OF THE INVENTION

The present invention generally relates to hair treatment formulations, in particular so-called conditioners, having an active ingredient combination for gentle and effective hair care.

### BACKGROUND OF THE INVENTION

The importance of care products with the longest possible effect has grown, not least of all due to the great stress on hair, for example, due to dyeing or permanent waves and also due to cleaning of hair with shampoos and due to environmental pollution. Such care products have an influence on the natural structure and properties of hair. For example, the wet and dry combability of hair, the hold and body of hair, can be optimized or the hair may be protected from increased split ends using appropriate care products.

It has therefore long been customary to subject hair to special after-treatments in which the hair is treated with special active ingredients, for example, quaternary ammonium salts or special polymers, usually in the form of a rinse. These treatments result in improved combability, hold and body of hair while reducing the amount of split ends, depending on the formulation.

Use of special oligopeptides to care for keratinic fibers is known from WO 2010/026010 A1, for example, in which oligopeptides in particular having the sequence Tyr-Glu-Glu-Glu-Ile-Arg-Val-Leu are disclosed as being particularly suitable for hair care.

There is still a need for active ingredients and/or active ingredient combinations for cosmetic formulations having good care properties and good biodegradability. In particular in formulations including dyes and/or electrolytes, there is a demand for additional care ingredients that can be incorporated easily into known formulations with no problem, where their effect is not diminished due to incompatibilities with other ingredients.

Furthermore, other desirable features and characteristics of the present invention will become apparent from the subsequent detailed description of the invention and the appended claims, taken in conjunction with this background of the invention.

### BRIEF SUMMARY OF THE INVENTION

A hair treatment formulation includes, based on its weight: 0.0001% to 10% at least one oligopeptide having at least one Glu-Glu-Glu amino acid sequence

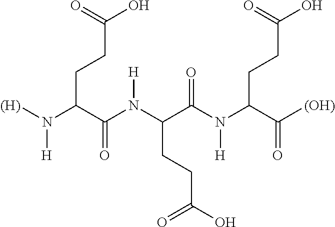

wherein the amino group may be present in free or protonated form and the carboxyl groups may be present in free or deprotonated form; 0.0001% to 10% at least one proteolipid of formula (I)

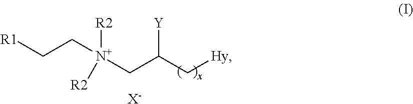

wherein R1 stands for a linear hydrocarbon radical with 8 to 22 carbon atoms, R2 stands for —(CH₂)_{y}—CH₃ where y=0 to 22, Y stands for —H or —OH, X⁻ stands for a physiologically tolerable anion, Hy stands for an oligopeptide radical from keratin hydrolysate and x stands for values from 0 to 22.

A method is also provided for improving at least one of the properties: tensile strength of keratinic fibers, in particular human hair; stabilization of the moisture balance of keratinic fibers, in particular human hair; combability of keratinic fibers, in particular human hair; delay in the aging process of keratinic fibers, in particular human hair; restructurability of keratinic fibers, in particular human hair during and after the permanent wave process; reduction in the decline in elasticity of keratinic fibers, in particular human hair when damaged due to atmospheric effects. A hair treatment formulation including, based on weight a) 0.0001% to 10% at least one oligopeptide having at least one Glu-Glu-Glu amino acid sequence

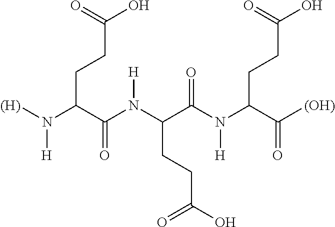

wherein the amino group may be present in free or protonated form and the carboxyl groups may be present in free or deprotonated form; and b) 0.0001% to 10% by weight of at least one proteolipid of formula (I)

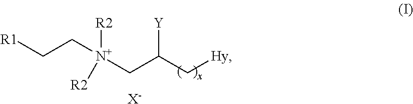

where R1 stands for a linear hydrocarbon radical with 8 to 22 carbon atoms, R2 stands for —(CH₂)_{y}—CH₃ where y=0 to 22, Y stands for —H or —OH, X⁻ stands for a physiologically tolerable anion, Hy stands for an oligopeptide radical from keratin hydrolysate, and x stands for values from 0 to 22, is applied to the keratinic fibers, left there for 5 seconds to 10 minutes and then rinsed out.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It has been found that proteolipids and certain oligopeptides have a mutually enhancing positive effect on hair and hair follicles.

A first subject matter of the present invention is hair treatment formulations including, each based on their weight:
a. 0.0001% to 10% by weight at least one oligopeptide having at least one Glu-Glu-Glu amino acid sequence

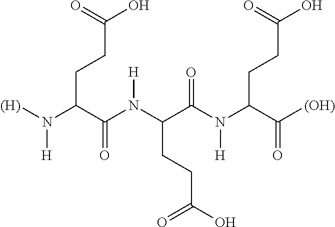

where the amino group may be free or protonated and the carboxyl groups may be free or deprotonated;
b. 0.0001% to 10% by weight at least one proteolipid of formula (I)

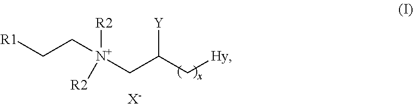

where
R1 stands for a linear hydrocarbon radical with 8 to 22 carbon atoms
R2 stands for —(CH₂)_{y}—CH₃, where y=0 to 22
Y stands for —H or —OH
X⁻ stands for physiologically tolerable anions,
Hy stands for an oligopeptide radical from keratin hydrolysate and
x stands for values from 0 to 22.

In this formula as well as in all the following formulas, the hydrogen atom in parentheses of the amino group, like the hydroxyl group in the oxygen function, means that the respective groups may be present as such (then this is an oligopeptide with the respective number of amino acids, as shown (in formula 3 above) or the amino acid sequence is present in an oligopeptide which also includes other amino acids—depending on where the additional amino acid(s) is/are bound and the components of the above formula shown in parentheses are replaced by the additional amino acid radical (radicals).

Hair treatment formulations in the sense of the present invention include, for example, hair shampoos, hair conditioners, conditioning shampoos, hair sprays, hair rinses, hair cures, hair packings, hair tonics, permanent wave fixative solutions, hair dye shampoos, hair dyeing formulations, hair setting formulations, hair styling preparations, blow drying lotions, foam solidifiers, hair gels, hair waxes or combinations thereof. In view of the fact that men are often unwilling to use several different products and/or to carry out a plurality of application steps, the hair treatment formulations according to the invention are those which men are already using anyway. Preferred formulations according to the invention therefore include shampoos, hair conditioning formulations or hair tonics. The hair treatment formulations according to the invention include—based on their weight-0.0001% to 10% by weight of at least one oligopeptide having at least one Glu-Glu-Glu amino acid sequence, i.e., at least three successive glutamic acids.

Oligopeptides in the sense of the present invention are condensation products of amino acids comprising at least 3 and max. 25 amino acids linked together by peptide bonds in the manner of an acid amide.

In the hair treatment formulations preferred according to the invention, the oligopeptide includes 5 to 15 amino acids, preferably 6 to 13 amino acids, especially preferably 7 to 12 amino acids and in particular 8, 9 or 10 amino acids.

Depending on whether additional amino acids are bound to the Glu-Glu-Glu sequence and depending on the type of amino acids there, the molecular weight of the oligopeptide included in the formulations according to the invention may vary. Hair treatment formulations preferred according to the invention are characterized in that the oligopeptide has a molecular weight of 650 to 3000 Da, preferably of 750 to 2500 Da, especially preferably from 850 to 2000 Da and in particular from 1000 to 1600 Da.

In summary, hair treatment formulations preferred to the invention are characterized in that the oligopeptide includes 5 to 15 amino acids, preferably 6 to 13 amino acids, especially preferably 7 to 12 amino acids and in particular 8, 9 or 10 amino acids and has a molecular weight of 650 to 3000 Da, preferably 750 to 2500 Da, especially preferably from 850 to 2000 Da and in particular from 1000 to 1600 Da. As can be seen from the preferred number of amino acids in the oligopeptides and the preferred molecular weight range, oligopeptides consisting not only of the three glutamic acids but also having other amino acids bound to this sequence are preferred for use. These additional amino acids are preferably selected from certain amino acids, whereas certain other representatives are less preferred according to the invention.

It is thus preferred if the oligopeptides used in the formulations according to the invention do not include any methionine. It is additionally preferred if the oligopeptides used in the formulations according to the invention do not include cysteine and/or cystine. It is also preferable if the oligopeptides used in the formulations according to the invention do not include any aspartic acid and/or asparagine. It is additionally preferred if the oligopeptides used in the formulations according to the invention do not include any serine and/or threonine. On the other hand, it is preferred according to the invention if the oligopeptides used in the formulations according to the invention include tyrosine. It is additionally preferred if the oligopeptides used in the formulations according to the invention include leucine. It is additionally preferred if the oligopeptides used in the formulations according to the invention include isoleucine. It is additionally preferred if the oligopeptides used in the formulations according to the invention include arginine. It is additionally preferred if the oligopeptides used in the formulations according to the invention include valine. Especially preferred oligopeptides and/or amino acid sequences included in the preferred oligopeptide are described below:

A particularly preferred oligopeptide additionally includes tyrosine which is preferably additionally bound by its acid function to the Glu-Glu-Glu sequence. Preferred hair treatment formulations according to the invention are therefore characterized in that the oligopeptide included therein has at least one Tyr-Glu-Glu-Glu amino acid sequence wherein the amino group may be in free or protonated form and the carboxyl groups may be in free or deprotonated form.

Another particularly preferred oligopeptide additionally includes isoleucine which is preferably bound by its amino function to the Glu-Glu-Glu sequence. Hair treatment formulations preferred according to the invention are therefore characterized in that the oligopeptide included in them has at least one Glu-Glu-Glu-Ile amino acid sequence wherein the amino group may be in free or protonated form and the carboxyl groups are in free or deprotonated form.

Oligopeptides including both of the aforementioned amino acids (tyrosine and isoleucine) are preferred according to the invention. Hair treatment formulations according to the invention in which the oligopeptide included in them has at least one Tyr-Glu-Glu-Glu-Ile amino acid sequence are particularly preferred, wherein the amino group may be present in free or protonated form and the carboxyl groups may be present in free or deprotonated form.

Additional preferred oligopeptides also include arginine which is preferably bound to isoleucine.

Hair treatment formulations according to any one of claims 1 to 6 are characterized in that the oligopeptide included in them has at least one Tyr-Glu-Glu-Glu-Ile-Arg amino acid sequence wherein the amino groups may be present in free or protonated form and the carboxyl groups may be present in free or deprotonated form.

Even more preferred oligopeptides additionally include valine which is preferably bound to the arginine. Additionally preferred hair treatment formulations according to the invention are therefore characterized in that the oligopeptide included in them has at least one Tyr-Glu-Glu-Glu-Ile-Arg-Val amino acid sequence wherein the amino groups may be present in free or protonated form and the carboxyl groups may be present in free or deprotonated form.

Even more preferred oligopeptides additionally include leucine which is preferably bound to the valine. Additionally preferred hair treatment formulations according to the invention are characterized in that the oligopeptide included in them has at least one Tyr-Glu-Glu-Glu-Ile-Arg-Val-Leu amino acid sequence wherein the amino groups may be present in free or protonated form and the carboxyl groups may be present in free or deprotonated form.

Particularly preferred oligopeptides additionally include leucine which is preferably bound to the tyrosine. Additionally preferred hair treatment formulations according to the invention are characterized in that the oligopeptide they include has at least one Leu-Tyr-Glu-Glu-Glu-Ile-Arg-Val-Leu amino acid sequence wherein the amino groups may be present in free or protonated form and the carboxyl groups may be present in free or deprotonated form.

Most especially preferably the formulations according to the invention include at least two oligopeptides which conform to the criteria specified above but are different from one another. Thus, hair treatment formulations including at least two different oligopeptides A and B, both of which include the Glu-Glu-Glu amino acid sequence are preferred.

Such mutually differing oligopeptides A and B correspond to one another in that they have three successive Glu amino acids in their amino acid sequence but have differences in the amino acids bound before and after those. Different peptides with a partial agreement, which may be much greater than that in the three amino acids mentioned above, are preferred.

Thus, additional preferred hair treatment formulations are characterized in that they include at least two different oligopeptides A and B, both of which include the Glu-Glu-Glu-Ile amino acid sequence.

Hair treatment formulations including at least two different oligopeptides A and B both of them including the Tyr-Glu-Glu-Glu amino acid sequence are also preferred.

Even more preferably hair treatment formulations are characterized in that they include at least two different oligopeptides A and B each of which includes the Glu-Glu-Glu-Ile-Arg amino acid sequence.

Additionally preferred hair treatment formulations also include at least two different oligopeptides A and B each of which has the Tyr-Glu-Glu-Glu-Ile amino acid sequence.

Most especially preferred hair treatment formulations are characterized in that they include at least two different oligopeptides A and B, both of which include the Glu-Glu-Glu-Ile-Arg amino acid sequence.

Most especially preferred hair treatment formulations also include at least two different oligopeptides A and B, both of which include the Tyr-Glu-Glu-Glu-Ile-Arg amino acid sequence.

There is preferably an even greater structural agreement in the oligopeptides. Thus hair treatment formulations including at least two different oligopeptides A and B both of which include the Glu-Glu-Glu-Ile-Arg-Val amino acid sequence are additional preferred embodiments of the present invention.

Hair treatment formulations including at least two different oligopeptides A and B both of which include the Tyr-Glu-Glu-Glu-Ile-Arg-Val amino acid sequence are also preferred.

Even more preferred hair treatment formulations according to the invention include at least two different oligopeptides A and B both of which include the Glu-Glu-Glu-Ile-Arg-Val-Leu amino acid sequence.

Even more preferred hair treatment formulations additionally include at least two different oligopeptides A and B both of which include the Tyr-Glu-Glu-Glu-Ile-Arg-Val-Leu amino acid sequence.

Particularly preferred hair treatment formulations are characterized in that they include at least two different oligopeptides A and B, wherein the oligopeptide A has the Leu-Tyr-Glu-Glu-Glu-Ile-Arg-Val-Leu amino acid sequence wherein the amino groups may be present in free or protonated form and the carboxyl groups may be present in free or deprotonated form, and the oligopeptide B has the Tyr-Glu-Glu-Glu-Ile-Arg-Val-Leu amino acid sequence wherein the amino groups may be present in free or protonated form and the carboxyl groups may be present in free or deprotonated form.

Most especially preferred formulations of this latter embodiment include—based on the weight of the formulation-0.00001 to 1% by weight oligopeptide A and 0.00001 to 1% by weight oligopeptide B. More preferred formulations of this latter embodiment include—based on the weight of the formulation-0.00005 to 0.1% by weight oligopeptide A and 0.00005 to 0.1% by weight oligopeptide B. Even more preferred formulations of this latter embodiment include, based on the weight of the formulation, 0.0001% to 0.01% by weight oligopeptide A and 0.0001% to 0.001% by weight oligopeptide B.

It has been found that use of the oligopeptides mentioned above imparts excellent properties to the hair treatment formulations according to the invention. The formulations according to the invention impart more stretching power to hair treated with them, which is manifested in higher tensile strength of the keratin fibers and in a lesser decline in elasticity, for example, in the case of damage due to atmospheric effects. In particular the especially preferred oligopeptides also stabilize the moisture balance of keratinic fibers so that combability is improved and the aging process is delayed.

Another advantage of the compositions according to the invention is that the shapeability and restructureability of keratinic fibers treated with them are improved. The oligopeptides which conform to the aforementioned conditions and are used within the context of the present invention may advantageously be obtained from keratinic materials. It is preferred according to the invention for these oligopeptides to be used in large amounts based on the total keratinic peptide content of the formulations.

It is most especially preferred for the greatest possible amount of all keratinic peptides included in the formulation to conform to the aforementioned conditions.

Preferred hair treatment formulations according to the invention are characterized in that at least 0.1% by weight, preferably at least 0.5% by weight, especially preferably at least 1% by weight, more preferably at least 2.5% by weight, even more preferably at least 5% by weight and in particular at least 10% by weight of all the keratinic peptides included in the formulation have the Glu-Glu-Glu amino acid sequence.

Additionally preferred hair treatment formulations according to the invention are characterized in that at least 0.1% by weight, preferably at least 0.5% by weight, especially preferably at least 1% by weight, additionally preferably at least 2.5% by weight, more preferably additionally at least 5% by weight and in particular at least 10% by weight of all the keratinic peptides included in the formulation have the Glu-Glu-Glu-Ile amino acid sequence.

Even more preferably preferred hair treatment formulations according to the invention are characterized in that at least 0.1% by weight, preferably at least 0.5% by weight, especially preferably at least 1% by weight, additionally preferably at least 2.5% by weight, more preferably additionally at least 5% by weight and in particular at least 10% by weight of all the keratinic peptides included in the formulation have the Tyr-Glu-Glu-Glu amino acid sequence.

Additionally preferred hair treatment formulations according to the invention are characterized in that at least 0.1% by weight, preferably at least 0.5% by weight, especially preferably at least 1% by weight, additionally preferably at least 2.5% by weight, more preferably additionally at least 5% by weight and in particular at least 10% by weight of all the keratinic peptides included in the formulation have the Tyr-Glu-Glu-Glu-Ile amino acid sequence.

Additionally preferred hair treatment formulations according to the invention are characterized in that at least 0.1% by weight, preferably at least 0.5% by weight, especially preferably at least 1% by weight, additionally preferably at least 2.5% by weight, more preferably additionally at least 5% by weight and in particular at least 10% by weight of all the keratinic peptides included in the formulation have the Tyr-Glu-Glu-Glu-Ile-Arg amino acid sequence.

Additionally preferred hair treatment formulations according to the invention are characterized in that at least 0.1% by weight, preferably at least 0.5% by weight, especially preferably at least 1% by weight, additionally preferably at least 2.5% by weight, more preferably additionally at least 5% by weight and in particular at least 10% by weight of all the keratinic peptides included in the formulation have the Tyr-Glu-Glu-Glu-Ile-Arg-Val amino acid sequence.

Additionally preferred hair treatment formulations according to the invention are characterized in that at least 0.1% by weight, preferably at least 0.5% by weight, especially preferably at least 1% by weight, additionally preferably at least 2.5% by weight, more preferably additionally at least 5% by weight and in particular at least 10% by weight of all the keratinic peptides included in the formulation have the Tyr-Glu-Glu-Glu-Ile-Arg-Val-Leu amino acid sequence.

The conditions defined above relate to the total amount of peptides originating from keratinic materials in the formulation according to the invention. In addition to the oligopeptides of keratinic origin, other peptides and/or protein hydrolysates may of course also be used, for example, from other native sources. For example, the additional use of wheat protein hydrolysates (see below) is preferred, for example.

The hair treatment formulations according to the invention include as the second essential ingredient—based on their weight-0.0001% to 10% by weight of at least one proteolipid of formula (I):

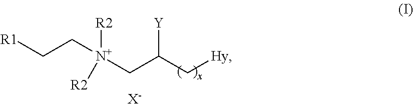

where
R1 stands for a linear hydrocarbon radical with 8 to 22 carbon atoms,
R2 stands for —(CH₂)_{y}—CH₃ where y=0 to 22,
Y stands for —H or —OH,
X⁻ stands for physiologically tolerable anions,
Hy stands for an oligopeptide radical from keratin hydrolysate and
x stands for values from 0 to 22.

Preferred radicals R1 are selected from —(CH₂)_{z}—CH₃ where z=1 to 15, and especially preferably z stands for the values 9, 11 or 13.

Preferred radicals R2 are selected from —(CH₂)_{z}—CH₃, where y=0 to 12, more preferably where y=0 to 8; y especially preferably stands for 0 (i.e., R2 is a methyl radical).

Y preferably stands for —OH. It is especially preferred when Y stands for —OH and x stands for 1. If Y=—H, then x preferably stands for a number from 1 to 7, especially preferably from 1 to 5, more preferably for 1 to 3 and in particular for 1.

Preferred hair treatment formulations according to the invention are characterized in that they include 0.0005% to 7.5% by weight, preferably 0.001% to 6% by weight, more preferably 0.005% to 5% by weight, more preferably 0.01% to 4% by weight and in particular 0.1% to 3% by weight of at least one proteolipid of formula (Ia):

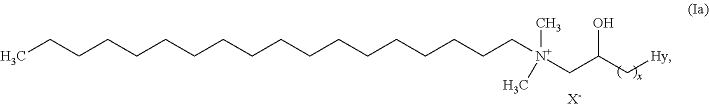

where X is selected from Cl⁻, Br⁻, ½SO₄²⁻, ⅓PO₄³⁻.

As already mentioned, Y may stand for —H. Preferred hair treatment formulations thus include 0.0005% to 7.5% by weight, preferably 0.001% to 6% by weight, more preferably 0.005% to 5% by weight, more preferably 0.01% to 4% by weight and in particular 0.1% to 3% by weight of at least one proteolipid of formula (Ib)

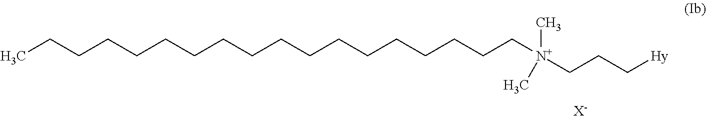

where X is selected from Cl⁻, Br⁻, ½SO₄²⁻, ½PO₄³⁻.

As already mentioned the use of proteolipids in which Y stands for —OH, R2 stands for a methyl group and x=1 are preferred. The Hy radical is an oligopeptide from keratin hydrolysate and preferably includes 3 to 12 amino acids.

Preferred hair treatment formulations thus include 0.0005% to 7.5% by weight, preferably 0.001% to 6% by weight, more preferably 0.005% to 5% by weight, more preferably 0.01% to 4% by weight and in particular 0.1% to 3% by weight of at least one proteolipid of formula (Ic)

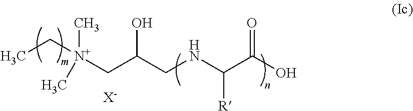

where
X is selected from Br, ½SO₄²⁻, ⅓PO₄³⁻,
m stands from numbers from 9 to 23,
n stands for the number of amino acids in the keratin hydrolysate, preferably for
numbers from 3 to 12,
R′ stands for the side chains of the amino acids in the keratin hydrolysate, where
R′ may be different in each recurring unit.

Preferably m stands for the number 9 (corresponding to a decyl radical attached to the N atom), 11 (dodecyl radical, lauryl radical), 13 (tetradecyl radical, myristyl radical), 15 (hexadecyl radical, palmityl radical) and extremely preferably 17 (octadecyl radical, stearyl radical). Corresponding compounds are available commercially, for example, under the INCI designation “steardimonium hydroxypropyl hydrolyzed keratin.” Also preferred is the use of compounds of formula (Ic) in which the long-chain radical on the N atom was obtained from natural fats via their fatty acid and/or fatty alcohol mixtures. One example preferred according to the invention is coconut fat which has a high lauric acid content so that the compound with the INCI designation “cocodimonium hydroxypropyl hydrolyzed keratin” that is available commercially and is preferred for use here is rich in compounds of formula (Ic) in which m stands for the number 11.

The formulations according to the invention may include additional active ingredients and auxiliary substances. These are described below.

The formulations according to the invention additionally include at least one emulsifier and/or one surfactant, wherein surface-active substances are referred to as surfactants or as emulsifiers, depending on the field of application, and are selected from anionic, cationic, zwitterionic, ampholytic and nonionic surfactants and emulsifiers.

Preferred hair treatment formulations according to the invention are characterized in that they include, based on their weight, 0.5% to 70% by weight, preferably 1% to 60% by weight and in particular 5% to 25% by weight anionic and/or nonionic and/or cationic and/or amphoteric surfactant(s).

Surface-active compounds with at least one quaternary ammonium group in the molecule and at least one —COO⁽⁻⁾ or —SO₃⁽⁻⁾ group referred to as zwitterionic surfactants and emulsifiers. Especially suitable zwitterionic surfactants and emulsifiers are the so-called betaines, such as N-alkyl-N,N-dimethylammonium glycinates, for example, cocoalkyldimethylammonium glycinate, N-acylaminopropyl-N,N-dimethylammonium glycinate, for example, cocoacylaminopropyldimethylammonium glycinate and 2-alkyl-3-carboxymethyl-3-hydroxyethylimidazolines each with 8 to 18 carbon atoms in the alkyl or acyl group as well as cocoacylaminoethylhydroxyethylcarboxylmethyl glycinate. A preferred zwitterionic surfactant is the fatty acid amide known by the INCI designation cocoamidopropylbetaine.

Ampholytic surfactants and emulsifiers are understood to refer to surface-active compounds which include at least one free amino group and at least one —COOH or —SO₃H group in addition to a C₈-C₂₄ alkyl or acyl group and are capable of forming internal salts. Examples of suitable ampholytic surfactants include N-alkylglycines, N-alkylaminopropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids, each one having app approx. 8 to 24 carbon atoms in the alkyl group. Especially preferred ampholytic surfactants include N-cocoalkylaminopropionate, cocoacylaminoethylaminopropionate and C₁₂-C₁₈ acylsarcosine.

Nonionic surfactants and emulsifiers include as the hydrophilic group, for example, a polyol group, a polyalkylene glycol ether group or a combination of polyol and polyglycol ether groups. In addition cationic surfactants of the following types can be used according to the invention: quaternary ammonium compounds, ester quats and amidoamines. Preferred quaternary ammonium compounds include ammonium halides, in particular chlorides and bromides, such as alkyltrimethylammonium chloride, dialkyldimethylammonium chloride and trialkylmethylammonium chloride. The long alkyl chains of these surfactants preferably have 10 to 18 carbon atoms such as cetyltrimethylammonium chloride, stearyltrimethylammonium chloride, distearyl-dimethylammonium chloride, lauryldimethylammonium chloride, lauryldimethylbenzyl-ammonium chloride and tricetylmethylammonium chloride. Other preferred cationic surfactants include the imidazolium compounds known under the INCI designations quaternium 27 and quaternium 83.

Especially preferred are formulations according to the invention which additionally include fatty alcohol(s) and/or fatty alcohol ethoxylate(s), preferably C₁₂₋₂₂ fatty alcohol(s) and/or C₁₂₋₂₂ fatty alcohol ethoxylate(s) with 10 to 30 EO units, especially preferably C₁₆₋₁₈ fatty alcohol(s) and/or C₁₆₋₁₈ fatty alcohol ethoxylate(s) with 12 to 20 EO units, preferably in amounts of 5% to 20% by weight, especially 7.5% to 17.5% by weight and in particular 10% to 15% by weight, each based on the weight of the formulation.

In summary, hair treatment formulations according to the invention which include, based on their weight, 0.1% to 20% by weight, preferably 0.25% to 17.5% by weight and in particular 5% to 15% by weight anionic surfactant(s), especially preferably fatty alcohol ether sulfates of the formula

H₃C—(CH₂)ₙ—(OCH₂CH₂)ₖ—OSO₃⁻M⁺

where n stands for values from 5 to 21, preferably from 7 to 19, especially preferably from 9 to 17 and in particular from 11 to 13, and k stands for values of 1, 2, 3, 4, 5, 6, 7, 8, 9 or 10, preferably for 1, 2 or 3 and in particular for 2, and M stands for a cation from the group Na⁺, K⁺, NH₄⁺, ½Mg²⁺, ½Zn²⁺, preferably for Na⁺.

Preferred hair treatment formulations according to the invention are additional characterized in that they also include amphoteric surfactant(s), preferably from the groups consisting of N-alkylglycines, N-alkylpropionic acids, N-alkylaminobutyric acids, N-alkyliminodipropionic acids, N-hydroxyethylN-alkylamidopropylglycines, N-alkyltaurines, N-alkylsarcosines, 2-alkylaminopropionic acids each preferably with approx. 8 to 24 carbon atoms in the alkyl group, alkylaminoacetic acids each with approx. 8 to 24 carbon atoms in the alkyl group, N-cocoalkylaminopropionate, cocosacylaminoethylaminopropionate, C₁₂-C₁₈ acylsarcosine, N-alkyl-N,N-dimethylammonium glycinates, for example, cocosalkyldimethylammonium glycinate, N-acyl-aminopropyl-N,N-dimethylammonium glycinate, for example, cocosacylaminopropyldimethylammonium glycinate, 2-alkyl-3-carboxymethyl-3-hydroxyethylimidazoline each with 8 to 18 carbon atoms in the alkyl or acyl group, cocosacylaminoethylhydroxyethylcarboxymethyl glycinate, the compounds known by the INCI designation cocoamidopropylbetaine, the compounds known by the INCI designation disodium cocoamphodiacetate, wherein preferred formulations include the amphoteric surfactant(s) in amounts of 1% to 15% by weight, preferably 2.5% to 12% by weight and in particular from 5% to 10% by weight, each based on the total formulation.

The formulations according to the invention may additionally include 0.01% to 10% by weight of at least one polymer from the group of cationic and/or amphoteric polymers as an additional optional component.

Cationic and/or amphoteric polymers are understood to be polymers which have in the main chain and/or side chain a group that may “temporarily” or “permanently” be cationic. “Permanently cationic” polymers according to the invention are those which have a cationic group regardless of the pH of the formulation. These are usually polymers which include a quaternary nitrogen atom, for example, in the form of an ammonium group. Preferred cationic groups are quaternary ammonium groups. In particular polymers in which the quaternary group is bound to a polymer main chain comprised of acrylic acid, methacrylic acid or derivatives thereof via a C₁₋₄ hydrocarbon group have proven to be particularly suitable.

In addition to or instead of cationic polymers, the formulations according to the invention may also include amphoteric polymers which additionally have at least one negatively charged group in the molecule and are also designated as zwitterionic polymers.

The polymer(s) is (are) preferably used within narrower quantity ranges. Thus, for example, formulations according to the invention including, based on their weight, 0.05% to 7.5% by weight, preferably 0.1% to 5% by weight, especially preferably 0.2% to 3.5% by weight and in particular 0.25% to 2.5% by weight amphoteric polymers are preferred.

Regardless of whether or not amphoteric polymers are included in the formulations, preferred formulations according to the invention are additionally characterized in that they include, based on their weight, 0.05% to 7.5% by weight, preferably 0.1% to 5% by weight, especially preferably 0.2 to 3.5% by weight and in particular 0.25% to 2.5% by weight cationic polymers.

In summary, hair treatment formulations according to the invention, which include, based on their weight, 0.05% to 7.5% by weight, preferably 0.1% to 5% by weight, especially preferably 0.2% to 3.5% by weight and in particular 0.25% to 2.5% by weight cationic polymer(s) are preferred, wherein preferred cationic polymer(s) is (are) selected from poly(methacryloyloxyethyltrimethylammonium chloride) (INCI: polyquaternium 37) and/or quaternized cellulose derivatives (INCI: polyquaternium 10) and/or cationic alkyl polyglycosides and/or cationized honey and/or cationic guar derivatives or polymeric dimethyldiallylammonium salts and copolymers with esters and amides of acrylic acid and methacrylic acid and/or copolymers of vinylpyrrolidone with quaternized derivatives of dialkylaminoalkyl acrylate and methacrylate and/or vinylpyrrolidone vinyl imidazolium methochloride copolymers and/or quaternized polyvinyl alcohol and/or its polyquaternium 2 and/or polyquaternium 7 and/or polyquaternium 17 and/or polyquaternium 18 and/or polyquaternium 24 and/or polyquaternium 27.

In addition to or instead of the cationic polymers, the formulations according to the invention may include amphoteric polymers.

In summary, the preferred formulations according to the invention are those in which the amphoteric polymer(s) is/are copolymers of at least one of the monomers trimethylammonium methyl acrylamide and/or trimethylammonium ethyl methacrylamide and/or trimethylammonium propyl acrylamide and/or trimethylammonium propyl methacrylamide and/or trimethylammonium methyl acrylamide and/or trimethylammonium methyl acrylate and/or trimethylammonium methyl methacrylate and/or trimethylammonium ethyl acrylate and/or ethyl dimethylammonium methyl acrylamide and/or ethyl dimethylammonium methyl methacrylamide and/or ethyl dimethylammonium propyl acrylamide and/or ethyl dimethylammonium propyl methacrylamide and/or ethyl dimethylammonium ethyl acrylamide and/or ethyl dimethylammonium methyl acrylate and/or ethyl dimethylammonium methyl methacrylate and/or ethyl dimethylammonium methyl acrylate wherein at least one monomers is acrylic acid and/or methacrylic acid and/or crotonic acid and/or 2-methylcrotonic acid.

Especially preferred amphoteric polymers according to the invention are:

copolymers of trimethylammonium ethyl acrylamide with acrylic acid
copolymers of trimethylammonium ethyl acrylamide with methacrylic acid
copolymers of trimethylammonium ethyl acrylamide with crotonic acid
copolymers of trimethylammonium ethyl acrylamide with 2-methylcrotonic acid
copolymers of trimethylammonium ethyl methacrylamide with acrylic acid
copolymers of trimethylammonium ethyl methacrylamide with methacrylic acid
copolymers of trimethylammonium ethyl methacrylamide with crotonic acid
copolymers of trimethylammonium ethyl methacrylamide with 2-methylcrotonic acid
copolymers of trimethylammonium propyl acrylamide with acrylic acid
copolymers of trimethylammonium propyl acrylamide with methacrylic acid
copolymers of trimethylammonium propyl acrylamide with crotonic acid
copolymers of trimethylammonium propyl acrylamide with 2-methylcrotonic acid
copolymers of trimethylammonium propyl methacrylamide with acrylic acid
copolymers of trimethylammonium propyl methacrylamide with methacrylic acid
copolymers of trimethylammonium propyl methacrylamide with crotonic acid
copolymers of trimethylammonium propyl methacrylamide with 2-methylcrotonic acid
copolymers of trimethylammonium ethyl acrylamide with acrylic acid
copolymers of trimethylammonium ethyl acrylamide with methacrylic acid
copolymers of trimethylammonium ethyl acrylamide with crotonic acid
copolymers of trimethylammonium ethyl acrylamide with 2-methylcrotonic acid
copolymers of trimethylammonium ethyl acrylate with acrylic acid
copolymers of trimethylammonium ethyl acrylate with methacrylic acid
copolymers of trimethylammonium ethyl acrylate with crotonic acid
copolymers of trimethylammonium ethyl acrylate with 2-methylcrotonic acid
copolymers of trimethylammonium ethyl methacrylate with acrylic acid
copolymers of trimethylammonium ethyl methacrylate with methacrylic acid
copolymers of trimethylammonium ethyl methacrylate with crotonic acid
copolymers of trimethylammonium ethyl methacrylate with 2-methylcrotonic acid
copolymers of trimethylammonium ethyl acrylate with acrylic acid
copolymers of trimethylammonium ethyl acrylate with methacrylic acid
copolymers of trimethylammonium ethyl acrylate with crotonic acid
copolymers of trimethylammonium ethyl acrylate with 2-methylcrotonic acid
copolymers of ethyl dimethylammonium ethyl acrylamide with acrylic acid
copolymers of ethyl dimethylammonium ethyl acrylamide with methacrylic acid
copolymers of ethyl dimethylammonium ethyl acrylamide with crotonic acid
copolymers of ethyl dimethylammonium ethyl acrylamide with 2-methylcrotonic acid
copolymers of ethyl dimethylammonium ethyl methacrylamide with acrylic acid
copolymers of ethyl dimethylammonium ethyl methacrylamide with methacrylic acid
copolymers of ethyl dimethylammonium ethyl methacrylamide with crotonic acid
copolymers of ethyl dimethylammonium ethyl methacrylamide with 2-methylcrotonic acid
copolymers of ethyl dimethylammonium propyl acrylamide with acrylic acid
copolymers of ethyl dimethylammonium propyl acrylamide with methacrylic acid
copolymers of ethyl dimethylammonium propyl acrylamide with crotonic acid
copolymers of ethyl dimethylammonium propyl acrylamide with 2-methylcrotonic acid
copolymers of ethyl dimethylammonium propyl methacrylamide with acrylic acid
copolymers of ethyl dimethylammonium propyl methacrylamide with methacrylic acid
copolymers of ethyl dimethylammonium propyl methacrylamide with crotonic acid
copolymers of ethyl dimethylammonium propyl methacrylamide with 2-methylcrotonic acid
copolymers of ethyl dimethylammonium ethyl acrylamide with acrylic acid
copolymers of ethyl dimethylammonium ethyl acrylamide with methacrylic acid
copolymers of ethyl dimethylammonium ethyl acrylamide with crotonic acid
copolymers of ethyl dimethylammonium ethyl acrylamide with 2-methylcrotonic acid
copolymers of ethyl dimethylammonium ethyl acrylate with acrylic acid
copolymers of ethyl dimethylammonium ethyl acrylate with methacrylic acid
copolymers of ethyl dimethylammonium ethyl acrylate with crotonic acid
copolymers of ethyl dimethylammonium ethyl acrylate with 2-methylcrotonic acid
copolymers of ethyl dimethylammonium ethyl methacrylate with acrylic acid
copolymers of ethyl dimethylammonium ethyl methacrylate with methacrylic acid
copolymers of ethyl dimethylammonium ethyl methacrylate with crotonic acid
copolymers of ethyl dimethylammonium ethyl methacrylate with 2-methylcrotonic acid
copolymers of ethyl dimethylammonium ethyl acrylate with acrylic acid
copolymers of ethyl dimethylammonium ethyl acrylate with methacrylic acid
copolymers of ethyl dimethylammonium ethyl acrylate with crotonic acid
copolymers of ethyl dimethylammonium ethyl acrylate with 2-methylcrotonic acid

As additional ingredients, the formulations according to the invention may especially preferably include one or more amino acids. Especially preferred amino acids for use according to the invention originate from the group comprised of glycine, alanine, valine, leucine, isoleucine, phenylalanine, tyrosine, tryptophan, proline, aspartic acid, glutamic acid, asparagine, glutamine, serine, threonine, cysteine, methionine, lysine, arginine, histidine, β-alanine, 4-aminobutyric acid (GABA), betaine, L-cystine (L-Cys), L-carnitine, L-citrulline, L-theanine, 3′,4′-dihydroxy-L-phenylalanine (L-dopa), 5′-hydroxy-L-tryptophan, L-homocysteine, S-methyl-L-methionine, S-allyl-L-cysteine sulfoxide (L-Alliin), L-trans-4-hydroxyproline, L-5-oxoproline (L-pyroglutamic acid), L-phosphoserine, creatine, 3-methyl-L-histidine, L-ornithine wherein both the individual amino acids as well as mixtures thereof may be used.

Preferred formulations according to the invention include one or more amino acids in narrower quantity ranges. Preferred hair treatment formulations according to the invention are characterized in that they include as the care substance, based on their weight, 0.01% to 5% by weight, preferably 0.02% to 2.5% by weight, especially preferably 0.05% to 1.5% by weight, more preferably 0.075% to 1% by weight and in particular 0.1% to 0.25% by weight amino acid(s), preferably from the group comprised of glycine and/or alanine and/or valine and/or lysine and/or leucine and/or threonine.

Another preferred group of ingredients of the formulations according to the invention includes vitamins, provitamins or vitamin precursors which are described below:

The group of substances identified as vitamin A includes retinol (vitamin A₁) as well as 3,4-didehydroretinol (vitamin A₂). β-Carotene is the provitamin of retinol. The vitamin A component that may be considered for use according to the invention includes, for example, vitamin A acid and esters thereof, vitamin A aldehyde and vitamin A alcohol as well as the esters thereof and the palmitate and acetate in amounts of 0.05% to 1% by weight, based on the total preparation.

Members of the vitamin B group or the vitamin B complex include, among others:

vitamin B₁ (thiamine)
vitamin B₂ (riboflavin)
vitamin B₃: this designation is frequently used to identify the compounds nicotinic acid and nicotinamide (niacinamide). Preferred according to the invention is nicotinamide which is preferably present in the formulations used according to the invention in amounts of 0.05 to 1% by weight, based on the total formulation.
vitamin B₅ (pantothenic acid, panthenol and pantolactone). Within the scope of this group, panthenol and/or pantolactone are preferably used. Derivatives of panthenol that can be used according to the invention include in particular the esters and ethers of panthenol as well as cationically derivatized panthenols. Individual representatives include, for example, panthenol triacetate, panthenol monoethyl ether and its monoacetate as well as the cationic panthenol derivatives disclosed in WO 92/13829. The aforementioned compounds of the vitamin B₅ type are present in the formulations according to the invention preferably in amounts of 0.05% to 10% by weight, based on the total formulation. Amounts of 0.1-5% by weight are especially preferred.
vitamin B₆ (pyridoxine and pyridoxamine and pyridoxal).

Vitamin C (ascorbic acid). Vitamin C is used in the formulation according to the invention preferably in amounts of 0.1% to 3% by weight based on the total formulation. Use in the form of the palmitic acid ester, the glucosides or phosphates may be preferred. Use in combination with tocopherols may also be preferred.

Vitamin E (tocopherols, in particular α-tocopherol). Tocopherol and its derivatives, in particular the esters such as the acetate, the nicotinate, the phosphate and the succinate, are preferably included in the formulations according to the invention in amounts of 0.05% to 1% by weight, based on the total formulation.

Vitamin F. The term “vitamin F” is usually understood to refer to essential fatty acids, in particular linoleic acid, linolenic acid and arachidonic acid.

Vitamin H. The compound (3aS,4S,6aR)-2-oxohexahydrothienol[3,4-d]imidazole-4-valeric acid is known as vitamin H, but in the meantime, the trivial name biotin has become established for this substance. Biotin is preferably included in the formulations according to the invention in amounts of 0.0001% to 1.0% by weight, in particular in amounts of 0.001% to 0.01% by weight.

In summary, the hair treatment formulations according to the invention, which additionally include as a care substance, based on their weight, 0.1% to 5% by weight, preferably 0.2% to 4% by weight, especially preferably 0.25% to 3.5% by weight, more preferably 0.5% to 3% by weight and in particular 0.5% to 2.5% by weight vitamins and/or provitamins and/or vitamin precursors, which are preferably assigned to the groups A, B, C, E, F and H, wherein preferred formulations include panthenol ((±)-2,4-dihydroxy-N-(3-hydroxypropyl)-3,3-dimethylbutyramide, provitamin B₅) and/or pantothenic acid (vitamin B₃, vitamin B₅) and/or niacin, niacinamide and/or nicotinamide (vitamin B₃) and/or L-ascorbic acid (vitamin C) and/or thiamine (vitamin B₁) and/or riboflavin (vitamin B₂, vitamin G) and/or biotin (vitamin B₇, vitamin H) and/or including folic acid (vitamin B₉, vitamin B_{c} or vitamin M) and/or vitamin B₆ and/or vitamin B₁₂.

To improve elasticity and strength of the internal structure of hair treated with the formulations according to the invention, the formulations according to the invention may include purine and/or purine derivatives. In particular the combination of purine and/or purine derivatives with ubiquinones and/or plastoquinones results in the fact that hair treated with the corresponding formulations yields higher measured values in differential thermal analysis and improved wet and dry combability, among other things.

Preferred formulations according to the invention include purine and/or purine derivatives in the narrower quantity ranges. Preferred cosmetic formulations according to the invention are characterized in that they include, based on their weight, 0.001% to 2.5% by weight, preferably 0.0025% to 1% by weight, especially preferably 0.005% to 0.5% by weight and in particular 0.01 to 0.1% by weight purine(s) and/or purine derivative(s).

Several representatives of purine, purines and purine derivatives are especially preferred according to the invention. Preferred hair treatment formulations are characterized in that they include as the care substance, based on their weight, 0.001% to 2.5% by weight, preferably 0.0025 to 1% by weight, especially preferably 0.005% to 0.5% by weight and in particular 0.01% to 0.1% by weight purine(s) and/or purine derivative(s), wherein preferred formulations include purine and/or purine derivative(s) from the group comprised of purine (R¹=R²=R³=R⁴=R⁵=R⁶=H), adenine (R¹=NH₂, R²=R³=R⁴=R⁵=R⁶=H), guanine (R¹=OH, R²=NH₂, R³=R⁴=R⁵=R⁶=H), uric acid (R¹=R²=R³=OH, R⁴=R⁵=R⁶=H), hypoxanthine (R¹=OH, R²=R³=R⁴=R⁵=R⁶=H), 6-purinethiol (R¹=SH, R²=R³=R⁴=R⁵=R⁶=H), 6-thioguanine (R¹=SH, R²=NH₂, R³=R⁴=R⁵=R⁶=H), xanthine (R¹=R²=OH, R³=R⁴=R⁵=R⁶=H), caffeine (R¹=R²=OH, R³=H, R⁴=R⁵=R⁶=CH₃), theobromine (R¹=R²=OH, R³=R⁴=H, R⁵=R⁶=CH₃), theophylline (R¹=R²=OH, R³=H, R⁴=CH₃, R⁵=CH₃, R⁶=H).

The type and amount of purine derivative may be varied, depending on the desired intended purpose of formulations according to the invention. Caffeine in particular has proven suitable in hair cosmetic formulations and may be used in shampoos preferably in amounts of 0.005% to 0.25% by weight, more preferably from 0.01% to 0.1% by weight and in particular from 0.01% to 0.05% by weight (each based on the shampoo).

As an additional ingredient, the formulations according to the invention may include at least one carbohydrate from the group of monosaccharides, disaccharides and/or oligosaccharides. Preferred hair treatment formulations according to the invention here are characterized in that they include, based on their weight, 0.01% to 5% by weight, preferably 0.05% to 4.5% by weight, especially preferably 0.1% to 4% by weight, more preferably 0.5% to 3.5% by weight and in particular 0.75% to 2.5% by weight carbohydrate(s) as the care substance, selected from monosaccharides, disaccharides and/or oligosaccharides.

As mentioned above, preferred formulations according to the invention include (an) amino acid(s).

Amino acids especially preferred for use according to the invention come from the group comprised of glycine, alanine, valine, leucine, isoleucine, phenylalanine, tyrosine, tryptophan, proline, aspartic acid, glutamic acid, asparagine, glutamine, serine, threonine, cysteine, methionine, lysine, arginine, histidine, β-alanine, 4-aminobutyric acid (GABA) betaine, L-cystine (L-Cyss), L-carnitine, L-citrulline, L-theanine, 3′,4′-dihydroxy-L-phenylalanine (L-dopa), 5′-hydroxy-L-tryptophan, L-homocysteine, S-methyl-L-methionine, S-allyl-L-cysteine sulfoxide (L-Alliin), L-trans-4-hydroxyproline, L-5-oxoproline (L-pyroglutamic acid), L-phosphoserine, creatine, 3-methyl-L-histidine, L-ornithine wherein both the individual amino acids and also mixtures thereof may be used.

Preferred formulations according to the invention include one or more amino acids in narrower quantity ranges. Cosmetic formulations preferred according to the invention here are characterized in that they additionally include 0.05% to 5% by weight, preferably 0.1% to 2.5% by weight, especially preferably 0.15% to 1% by weight and in particular 0.2% to 0.5% by weight amino acid(s), preferably (an) amino acid(s) from the group comprised of glycine and/or alanine and/or valine and/or lysine and/or leucine and/or threonine.

Taurine is another preferred care substance for use, which has activating properties. Hair treatment formulations that are preferred according to the invention include as the care substance, based on weight, 0.01% to 15% by weight, preferably 0.025% to 12.5% by weight, especially preferably 0.05% to 10% by weight, more preferably 0.1% to 7.5% by weight and in particular 0.5% to 5% by weight taurine (2-aminoethane sulfonic acid).

The additional use of bisabolol and/or bisabolol oxides in the formulation according to the invention is also preferred. Hair treatment formulations according to the invention, which additionally include 0.001% to 5% by weight, preferably 0.01% to 4% by weight, especially preferably 0.02 to 2.5% by weight and in particular 0.1 to 1.5% by weight bisabolol and/or oxides of bisabolol, preferably (−)-α-bisabolol, are preferred.

In addition to the oligopeptide(s), the hyaluronic acid ester(s) and optional additional ingredients, the formulations according to the invention may also include other substances which prevent, relieve or cure hair loss. It is advantageous in particular for these products to include active ingredients that stabilize the hair follicle. In summary, cosmetic formulations according to the invention, which additionally include, based on their weight, 0.001% to 5% by weight hair follicle stabilizing substances, in particular minoxidil and/or finasteride and/or ketoconazole, are preferred.

With additional anti-dandruff active ingredients (for example, climbazole, piroctone olamine or pyrithione zinc), the amount of fungus that causes dandruff is greatly reduced, the microbial flora is returned to its normal percentage composition and desquamation is reduced to the physiological extent. However, laboratory tests have proven that the different species of Pityrosporum ovale respond to the anti-dandruff active ingredients to different extents. To maximally combat all dandruff pathogens, a combination of antidandruff active ingredients is therefore the most successful.

In summary, the preferred hair treatment formulations according to the invention additionally include, based on their weight, 0.001% to 5% by weight antidandruff active ingredients, in particular piroctone olamine (1-hydroxy-4-methyl-6-(2,4,4-trimethylpentyl)pyridin-2(1H)-one, compound with 2-aminoethanol, 1:1) and/or pyrithione zinc and/or selenium sulfide and/or climbazole and/or salicylic acid or fumaric acid.

The formulations according to the invention may also include the active ingredients, additives and excipients known for such preparations. In many cases the formulations will include at least one surfactant, wherein in principle both anionic and zwitterionic, ampholytic, nonionic and cationic surfactants are suitable. In many cases, however, it has proven advantageous to select the surfactants from anionic, zwitterionic or nonionic surfactants. These surfactants were explained in detail further above.

In another preferred embodiment the formulations according to the invention may include emulsifiers (F). The formulations according to the invention preferably include the emulsifiers in amounts of 0.1-25% by weight, in particular 0.5-15% by weight, based on the total formulation.

The compositions according to the invention may preferably include at least one nonionic emulsifier with an HLB value of 8 to 18. Nonionic emulsifiers with an HLB value of 10 to 15 may be particularly preferred according to the invention.

It has also proven advantageous if, in addition to the polymer(s) from the group of cationic and/or amphoteric polymers, additional polymers (G) are included in the formulations according to the invention. In a preferred embodiment therefore additional polymers are added to the formulations according to the invention, wherein both anionic and nonionic polymers have proven effective.

In another embodiment, the formulations according to the invention may include nonionic polymers (G4).

The additional polymers (G) are preferably present in the formulations according to the invention in amounts of 0.05% to 10% by weight, based on the total formulation. Amounts of 0.1% to 5%, in particular 0.1% to 3% by weight are especially preferred.

In addition, in a preferred embodiment of the invention, a formulation according to the invention may also include UV filters (I). The UV filters to be used according to the invention are not subject to any general restrictions with regard to their structure and their physical properties. Instead, all the UV filters which can be used in the cosmetic field and whose absorption maximum is in the UVA range (315-400 nm), in the UVB range (280-315 nm) or in the UVC range (<280 nm), are suitable. UV filters with an absorption peak in the UVB range, in particular in the range of approx. 280 to 30 nm, are especially preferred.

The UV filters (I) are usually included in the formulations according to the invention in amounts of 0.1-5% by weight, based on the total formulation. Amounts of 0.4-2.5% by weight are preferred.

Finally, the formulations according to the invention may also include plant extracts (L).

These extracts are usually prepared by extraction of the entire plant, but in individual cases it may also be preferable to prepare the extracts exclusively from the flowers and/or leaves of the plants.

Especially preferred according to the invention are the extracts of green tea, oak bark, stinging nettle, witch hazel, hops, henna, chamomile, burdock root, horsetail, hawthorne, lime blossom, almond, aloe vera, pine needle, horse chestnut, sandalwood, juniper, coconut, mango, apricot, lemon, wheat, kiwi, melon, orange, grapefruit, sage, rosemary, birch, mallow, cuckoo flower/lady's smock, creeping thyme, yarrow, thyme, melissa, harrow, colt's foot, cranberry, meristem, ginseng and ginger root.

In addition, it may be preferable to use mixtures of several different plant extracts, in particular two in the formulations according to the invention. In addition, it may prove advantageous if penetration aids and/or swelling formulations (M) are present in the formulations according to the invention. These include, for example, urea and urea derivatives, guanidine and its derivatives, arginine and its derivatives, water glass, imidazole and its derivatives, histidine and its derivatives, benzyl alcohol, glycerol, glycol and glycol ethers, propylene glycol and propylene glycol ethers, for example, propylene glycol monoethyl ether, carbonates, bicarbonates, diols and triols and in particular 1,2-diols and 1,3-diols such as, for example, 1,2-propanediol, 1,2-pentanediol, 1,2-hexanediol, 1,2-dodecanediol, 1,3-propanediol, 1,6-hexanediol, 1,5-pentanediol, 1,4-butanediol.

Silicones are a particularly preferred group of ingredients.

Preferred formulations according to the invention are characterized in that they include at least one silicone.

Particularly preferred formulations according to the invention preferably include the silicone(s) in amounts of 0.1 to 10% by weight, preferably 0.25 to 7% by weight and in particular from 0.5 to 5% by weight, each based on the total formulation.

Especially preferred formulations according to the invention are characterized in that they include at least one silicone of the formula Si-I

(CH₃)₃Si—[O—Si(CH₃)₂]ₓ—O—Si(CH₃)₃ (Si-I),

where x stands for a number from 0 to 100, preferably from 0 to 50, more preferably from 0 to 20 and in particular 0 to 10.

These silicones are designated as dimethicone according to the INCI nomenclature. The following compounds are preferably used as the silicone of formula Si-I within the scope of the present invention:

(CH₃)₃Si—O—Si(CH₃)₃ (CH₃)₃Si—[O—(CH₃)₂Si]₁₁—O—Si(CH₃)₃
(CH₃)₃Si—O—(CH₃)₂Si—O—Si(CH₃)₃ (CH₃)₃Si—[O—(CH₃)₂Si]₁₂—O—Si(CH₃)₃
(CH₃)₃Si—[O—(CH₃)₂Si]₂—O—Si(CH₃)₃ (CH₃)₃Si—[O—(CH₃)₂Si]₁₃—O—Si(CH₃)₃
(CH₃)₃Si—[O—(CH₃)₂Si]₃—O—Si(CH₃)₃ (CH₃)₃Si—[O—(CH₃)₂Si]₁₄—O—Si(CH₃)₃
(CH₃)₃Si—[O—(CH₃)₂Si]₄—O—Si(CH₃)₃ (CH₃)₃Si—[O—(CH₃)₂Si]₁₅—O—Si(CH₃)₃
(CH₃)₃Si—[O—(CH₃)₂Si]₅—O—Si(CH₃)₃ (CH₃)₃Si—[O—(CH₃)₂Si]₁₆—O—Si(CH₃)₃
(CH₃)₃Si—[O—(CH₃)₂Si]₆—O—Si(CH₃)₃ (CH₃)₃Si—[O—(CH₃)₂Si]₁₇—O—Si(CH₃)₃
(CH₃)₃Si—[O—(CH₃)₂Si]₇—O—Si(CH₃)₃ (CH₃)₃Si—[O—(CH₃)₂Si]₁₈—O—Si(CH₃)₃
(CH₃)₃Si—[O—(CH₃)₂Si]₈—O—Si(CH₃)₃ (CH₃)₃Si—[O—(CH₃)₂Si]₁₉—O—Si(CH₃)₃
(CH₃)₃Si—[O—(CH₃)₂Si]₉—O—Si(CH₃)₃ (CH₃)₃Si—[O—(CH₃)₂Si]₂₀—O—Si(CH₃)₃
(CH₃)₃Si—[O—(CH₃)₂Si]₁₀—O—Si(CH₃)₃
where (CH₃)₃Si—O—Si(CH₃)₃, (CH₃)₃Si—O—(CH₃)₂Si—O—Si(CH₃)₃ and/or (CH₃)₃Si—[O—(CH₃)₂Si]₂—O—Si(CH₃)₃ are especially preferred.

Mixtures of the aforementioned silicones may of course also be present in the formulations according to the invention.

Preferred silicones for use according to the invention have a viscosity of 0.2 to 2 mm²s⁻¹ at 20° C., but silicones with a viscosity of 0.5 to 1 mm²s⁻¹ are especially preferred.

Especially preferred formulations according to the invention include one or more amino-functional silicones.

Preferred formulations according to the invention are characterized in that they include an amino-functional silicone of the formula Si-II

R′ₐG₃₋ₐ-Si(OSiG₂)ₙ-(OSiG_{b}R′_{2-b})ₘ—O—SiG₃₋ₐ-R′ₐ (Si-II),

wherein:
G is —H, a phenyl group, —OH, —O—CH₃, —CH₃, —O—CH₂CH₃, —CH₂CH₃, —O—CH₂CH₂CH₃,
—CH₂CH₂CH₃, —O—CH(CH₃)₂, —CH(CH₃)₂, —O—CH₂CH₂CH₂CH₃, CH₂CH₂CH₂CH₃,
—O—CH₂CH(CH₃)₂, —CH₂CH(CH₃)₂, —O—CH(CH₃)CH₂CH₃, —CH(CH₃)CH₂CH₃, —O—C(CH₃)₃,
—C(CH₃)₃;
a stands for a number between 0 and 3, in particular 0;
b stands for a number between 0 and 1, in particular 1,
m and n are numbers, whose sum (m+n) is between 1 and 2000, preferably between 50 and 150, where n preferably assumes values from 0 to 1999 and in particular from 49 to 149, and m preferably assumes values from 1 to 2000, in particular from 1 to 10,
R′ is a monovalent radical selected from
   -Q-N(R″)—CH₂—CH₂—N(R″)²
   -Q-N(R″)₂
   -Q-N⁺(R″)₃A⁻
   -Q-N⁺H(R″)₂A⁻
   -Q-N⁺H₂(R″)A⁻
   -Q-N(R″)—CH₂—CH₂—N⁺R″H₂A⁻,
wherein each Q stands for a chemical bond, —CH₂—, —CH₂—CH₂—, —CH₂CH₂CH₂—, —C(CH₃)₂—, —CH₂CH₂CH₂CH₂—, —CH₂C(CH₃)₂—, —CH(CH₃)CH₂CH₂—,
R″ stands for the same or different radicals from the group —H, phenyl, benzyl, —CH₂—CH(CH₃)Ph, the C₁₋₂₀ alkyl radicals, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃ and A represents an anion, which is preferably selected from chloride, bromide, iodide or methosulfate.

Formulations according to the invention that are particularly preferred are characterized in that they include at least one amino-functional silicone of the formula Si-IIa

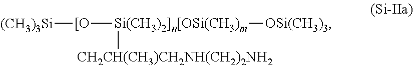

wherein m and n are numbers, the sum of which (m+n) is between 1 and 2000, preferably between 50 and 150, where n preferably assumes values from 0 to 1999 and in particular from 49 to 149 and m preferably assumes values from 1 to 2000 and in particular from 1 to 10.

According to the INCI declaration, these silicones are designated as trimethylsilylamodimethicone.

Formulations according to the invention including an amino-functional silicone of the formula Si-IIb are also particularly preferred

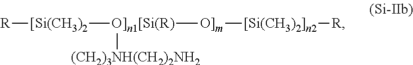

wherein R stands for —OH, —O—CH₃ or a —CH₃ group and m, n1 and n2 are numbers whose sum (m+n1+n2) amounts to between 1 and 2000, preferably between 50 and 150, where the sum (n1+n2) preferably assumes values from 0 to 1999 and in particular from 49 to 149, and m preferably assumes values from 1 to 2000, in particular from 1 to 10.

These silicones are designated as amodimethicones according to the INCI designation.

Regardless of which amino-functional silicones are used, formulations according to the invention including an amino-functional silicone, whose amine number is higher than 0.25 meq/g, preferably higher than 0.3 meq/g and in particular higher than 0.4 meq/g are preferred. The amine number stands for milliequivalents of amine per gram of amino-functional silicone. It can be determined by titration and is also given in units of mg KOH/g.

Preferred formulations according to the invention are characterized in that they include, based on their weight, 0.01 to 10% by weight, preferably 0.1 to 8% by weight, especially preferably 0.25 to 7.5% by weight and in particular 0.5 to 5% by weight amino-functional silicone(s).

The silicones described above have a backbone constructed of Si—O—Si units. These Si—O—Si units may of course also be interrupted by carbon chains. Corresponding molecules are accessible by chain lengthening reactions and are used preferably in the form of silicone-in-water emulsions.

Formulations that are also preferred according to the invention are characterized in that they include at least one silicone of the formula Si-IV

R₃Si—[O—SiR₂]ₓ—(CH₂)ₙ—[O—SiR₂]_{y}—O—SiR₃ (Si-IV),

where R stands for the same or different radicals from the group —H, phenyl, benzyl, —CH₂—CH(CH₃)Ph, the C₁₋₂₀ alkyl radicals, preferably —CH₃, —CH₂CH₃, —CH₂CH₂CH₃, —CH(CH₃)₂,
—CH₂CH₂CH₂H₃, —CH₂CH(CH₃)₂, —CH(CH₃)CH₂CH₃, —C(CH₃)₃, x and/or y stand(s) for a number from 0 to 200, preferably from 0 to 10, more preferably from 0 to 7 and in particular 0, 1, 2, 3, 4, 5 or 6 and n stands for a number from 0 to 10, preferably from 1 to 8 and in particular for 2, 3, 4, 5 or 6.

The silicones are preferably water soluble. Preferred formulations according to the invention are characterized in that they include at least one water-soluble silicone. For aesthetic reasons, “clear” products are often preferred by consumers. Preferred hair treatment formulations according to the invention are therefore characterized in that they are transparent and/or translucent. Transparent or translucent within the context of present invention are understood to refer to a composition having an NTU value of less than 100. The NTU value or nephelometric turbidity unit is a unit used in water processing for turbidity measurements in liquids; it is the unit of a turbidity of a liquid measured with a calibrated nephelometer.

The formulations according to the invention have advantageous properties and also impart advantageous properties to the hair treated with them. Advantages going beyond the advantages of the oligopeptides and/or proteolipids alone are observed in particular in the treatment of hair and scalp. Thus hair treatment formulations according to the invention increase the elasticity of hair treated with them and lead to an inner-structural strengthening of the hair fibers, which is reflected in higher melting temperatures in differential thermal analysis, for example.

There is also an improvement in wet and dry combability as well as preventing premature split ends in the treated hair. The formulations according to the invention also cause an increase in elasticity and surprisingly in sebum-regulating effects on the skin and in particular the scalp. The visual impression of “oily” skin or hair is thus prevented and/or diminished.

Another subject matter of the present invention is a method for improving at least one of the properties:
tensile strength of keratinic fibers, in particular human hair;
stabilization of the moisture balance of keratinic fibers, in particular human hair;
combability of keratinic fibers, in particular human hair;
delay in the aging process of keratinic fibers, in particular human hair;
restructurability of keratinic fibers, in particular human hair during and after a permanent wave treatment;
reduction in the decline in elasticity of keratinic fibers, in particular human hair in the case of damage due to atmospheric effects
   wherein a hair treatment formulation including, each based on weight:
a. 0.0001% to 10% by weight of at least one oligopeptide having at least one Glu-Glu-Glu amino acid

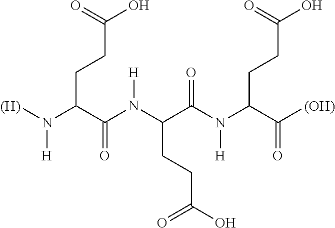

wherein the amino group may be present in free or protonated form and the carboxyl groups may be present in free or deprotonated form,
b. 0.0001% to 10% by weight of at least one proteolipid of formula (I)

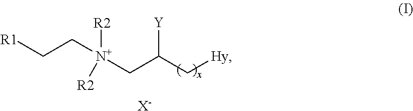

where

R1 stands for a linear hydrocarbon radical with 8 to 22 carbon atoms,
R2 stands for —(CH₂)_{y}—CH₃, wherein y=0 to 22,
Y stands for —H or —OH,
X⁻ stands for a physiologically tolerable anion,
Hy stands for an oligopeptide radical from keratin hydrolysate and
x stands for values from 0 to 22,
   when applied to the keratinic fibers, left there for 5 seconds to 10 minutes and then rinsed out.

With regard to additional preferred embodiments of the method according to the invention, what was said about the formulations according to the invention also applies here, mutatis mutandis.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A hair treatment formulations including, based on its weight:
a) 0.0001% to 10% by weight at least one oligopeptide having at least one Glu-Glu-Glu amino acid sequence 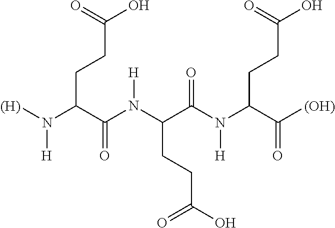
wherein the amino group may be present in free or protonated form and the carboxyl groups may be present in free or deprotonated form; and
b) 0.0001% to 10% by weight at least one proteolipid of formula (I) 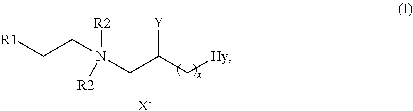
wherein
R1 stands for a linear hydrocarbon radical with 8 to 22 carbon atoms,
R2 stands for —(CH₂)_{y}—CH₃ where y=0 to 22,
Y stands for —H or —OH,
X⁻ stands for a physiologically tolerable anion,
Hy stands for an oligopeptide radical from keratin hydrolysate and
x stands for values from 0 to 22.

2. Hair treatment formulation according to claim 1, wherein the oligopeptide includes 5 to 15 amino acids and has a molecular weight of 650 to 3000 Da.

3. Hair treatment formulation according to claim 1, wherein the oligopeptide has Tyr-Glu-Glu-Glu-Ile 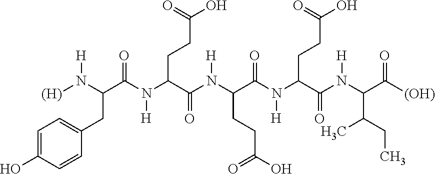
wherein the amino group may be present in free or protonated form and the carboxyl groups may be present in free or deprotonated form.

4. Hair treatment formulation according to claim 1, wherein the oligopeptide has at least one Tyr-Glu-Glu-Glu-Ile-Arg-Val-Leu amino acid sequence 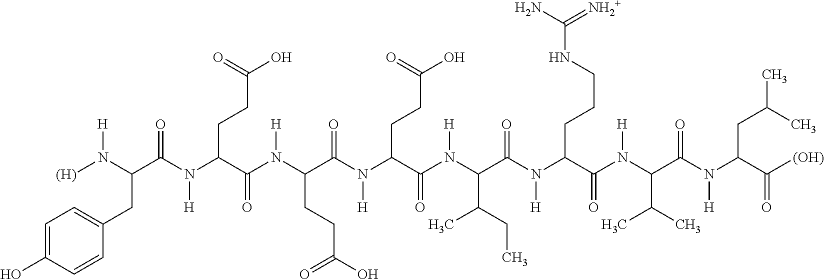
wherein the amino group may be present in free or protonated form and the carboxyl groups may be present in free or deprotonated form.

5. Hair treatment formulation according to claim 1, wherein the formulation includes 0.0005% to 7.5% by weight at least one proteolipid of formula (Ia); 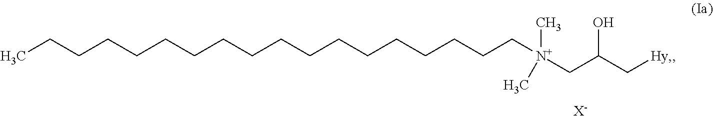
wherein X is selected from Br, ½SO₄²⁻, and ¼PO₄³⁻.

6. Hair treatment formulation according to claim 1, wherein the formulation includes 0.0005% to 7.5% by weight at least one proteolipid of formula (Ib) 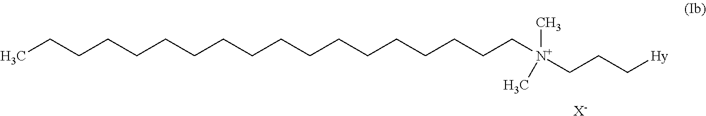
wherein X is selected from Cl⁻, Br⁻, ½SO₄²⁻, and ⅓PO₄³⁻.

7. Hair treatment formulation according to claim 1, wherein the formulation includes 0.0005% to 7.5% by weight at least one proteolipid of formula (Ic) 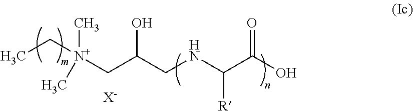
wherein
X is selected from Cl⁻, Br⁻, ½SO₄²⁻, and ⅓PO₄³⁻,
m stands from numbers from 9 to 23,
n is 3 to 12, and
R′ stands for the side chains of the amino acids in the keratin hydrolysate, wherein R′ may be different in each recurring unit.

8. Hair treatment formulation according to claim 1, wherein the formulation includes based on its weight 0.5% to 70% by weight anionic and/or nonionic and/or cationic and/or amphoteric surfactant(s).

9. Hair treatment formulation according to claim 1, wherein the formulation includes 0.05% to 7.5% by weight one or more cationic polymer selected from the group consisting of
poly(methacryloyloxyethyltrimethylammonium chloride) having the INCI name polyquaternium 37,
quaternized cellulose derivative having the INCI name polyquaternium 10,
cationic alkyl polyglycosides,
cationized honey,
cationic guar derivatives,
polymeric dimethyldiallylammonium salts and their copolymers with esters and amides of acrylic acid and methacrylic acid,
copolymers of vinylpyrrolidone with quaternized derivatives of dialkylaminoalkyl acrylate and methacrylate,
vinylpyrrolidone-vinyl imidazolium methochloride copolymers,
quaternized polyvinyl alcohol,
polyquaternium 2,
polyquaternium 7,
polyquaternium 17,
polyquaternium 18,
polyquaternium 24, and
polyquaternium 27.

10. A method for improving at least one of the properties:
tensile strength of keratinic fibers, in particular human hair;
stabilization of the moisture balance of keratinic fibers, in particular human hair;
combability of keratinic fibers, in particular human hair;
delay in the aging process of keratinic fibers, in particular human hair;
restructurability of keratinic fibers, in particular human hair during and after the permanent wave process;
reduction in the decline in elasticity of keratinic fibers, in particular human hair when damaged due to atmospheric effects, includes:
applying to the keratinic fibers a hair treatment formulation including, based on weight
a) 0.0001% to 10% by weight at least one oligopeptide having at least one Glu-Glu-Glu amino acid sequence 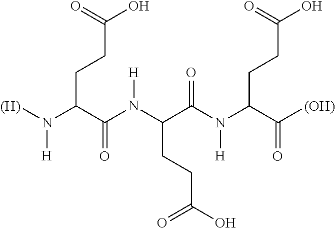
wherein the amino group may be present in free or protonated form and the carboxyl groups may be present in free or deprotonated form, and
b) 0.0001% to 10% by weight at least one proteolipid of formula (I) 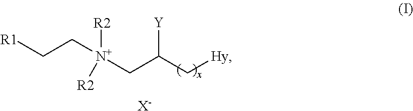
wherein
R1 stands for a linear hydrocarbon radical with 8 to 22 carbon atoms,
R2 stands for —(CH₂)_{y}—CH₃ wherein y=0 to 22,
Y stands for —H or —OH,
X⁻ stands for a physiologically tolerable anion,
Hy stands for an oligopeptide radical from keratin hydrolysate and
x stands for values from 0 to 22, and
leaving the hair treatment in the keratinic fibers for 5 seconds to 10 minutes before rinsing it out.


SEQUENCE LISTING (7 sequences)

SEQ ID NO: 1 - protein, 3 aa, Artificial
  Peptide
EEE

SEQ ID NO: 2 - protein, 4 aa, Artificial
  Peptide
YEEE

SEQ ID NO: 3 - protein, 4 aa, Artificial
  Peptide
EEEI

SEQ ID NO: 4 - protein, 6 aa, Artificial
  Peptide
YEEEIR

SEQ ID NO: 5 - protein, 7 aa, Artificial
  Peptide
YEEEIRV

SEQ ID NO: 6 - protein, 8 aa, Artificial
  Peptide
YEEEIRVL

SEQ ID NO: 7 - protein, 9 aa, Artificial
  Peptide
LYEEEIRVL